(19) United States
(12) Patent Application Publication
(10) Pub. No.: US 2014/0276060 A1
(43) Pub. Date: Sep. 18, 2014

(54) ULTRASONIC MEASURING DEVICE, ULTRASONIC IMAGE DEVICE, AND MEASURING METHOD

(71) Applicant: SEIKO EPSON CORPORATION, Tokyo (JP)

(72) Inventors: Masaki HAYASHI, Shiojiri (JP); Ryoki WATANABE, Suwa (JP)

(73) Assignee: SEIKO EPSON CORPORATION, Tokyo (JP)

(21) Appl. No.: 14/208,022

(22) Filed: Mar. 13, 2014

(30) Foreign Application Priority Data
Mar. 15, 2013 (JP) 2013-053229

(51) Int. Cl. A61B 8/14

(52) U.S. Cl. CPC A61B 8/14
USPC 600/443; 600/437

(57) ABSTRACT

An ultrasonic measuring device can acquire inclination information regarding an ultrasonic probe and notify the user if the ultrasonic probe is inclined. The ultrasonic measuring device includes an emission unit that performs ultrasound emission processing, a reception unit that performs ultrasonic echo reception processing, and a processing unit that performs ultrasonic measurement control processing. The processing unit acquires inclination information regarding the ultrasonic probe based on a reception signal resulting from ultrasonic echoes from an interface between the test subject and an ultrasonic measurement sheet, or resulting from ultrasonic echoes from the ultrasonic measurement sheet.

### BACKGROUND

1. Technical Field

The present invention relates to an ultrasonic measuring device, an ultrasonic image device, a measuring method, and the like.

2. Related Art

An ultrasonic measuring device and an ultrasonic image device are examples of a device that acquires subject images or the like by emitting ultrasonic waves toward a subject and receiving reflected waves from interfaces between materials that have different acoustic impedances inside the subject. These devices can be used to measure subcutaneous fat thickness, muscle thickness, and the like.

For example, JP-A-2009-77754 discloses a technique in which a B mode image is acquired by manually moving an ultrasonic probe along the surface of a test subject and detecting the translation amount and rotation amount with an optical position sensor and an angular velocity sensor, and the subcutaneous fat thickness is determined.

JP-A-2009-77754 is an example of related art.

### SUMMARY

However, with this technique, when measuring a highly-curved portion such as an arm or a leg, there are problems such as the ultrasonic probe tending to become inclined relative to the test subject, thus making it difficult to perform accurate measurement.

According to several aspects of the invention, it is possible to provide an ultrasonic measuring device, an ultrasonic image device, a measuring method, and the like that can acquire inclination information regarding the ultrasonic probe and notify the user if the ultrasonic probe is inclined.

A first aspect of the invention relates to an ultrasonic measuring device including: an emission unit that performs ultrasound emission processing; a reception unit that performs ultrasonic echo reception processing; and a processing unit that performs ultrasonic measurement control processing, wherein the processing unit acquires inclination information regarding an ultrasonic probe based on a reception signal resulting from ultrasonic echoes from an interface between a test subject and an ultrasonic measurement sheet, or resulting from ultrasonic echoes from the ultrasonic measurement sheet.

According to this aspect of the invention, the processing unit can acquire inclination information regarding the ultrasonic probe, thus making it possible to notify the user of whether or not the ultrasonic probe is inclined. As a result, the user can hold the ultrasonic probe without being inclined relative to the test subject while performing measurement.

Also, in the first aspect of the invention, the processing unit may acquire depth information regarding the interface in ultrasonic measurement based on the reception signal, and acquire the inclination information based on the depth information.

According to this configuration, the processing unit can acquire inclination information regarding the ultrasonic probe by acquiring depth information regarding the interface between the test subject and the ultrasonic measurement sheet in ultrasonic measurement.

Also, in the first aspect of the invention, the processing unit may acquire the inclination information by performing processing for comparing the depth information with reference thickness information of the ultrasonic measurement sheet.

According to this configuration, the processing unit can acquire inclination information regarding the ultrasonic probe based on depth information and reference thickness information of the ultrasonic measurement sheet.

Also, in the first aspect of the invention, the depth information may be depth information in an ultrasound emission direction from a face of the ultrasonic measurement sheet that opposes the ultrasonic probe to the interface between the ultrasonic measurement sheet and the test subject.

According to this configuration, if the ultrasonic probe is inclined relative to the test subject, the depth based on the depth information is greater than the reference thickness of the ultrasonic measurement sheet, and therefore the processing unit can acquire inclination information regarding the ultrasonic probe by comparing the depth information with the reference thickness information of the ultrasonic measurement sheet.

Also, in the first aspect of the invention, the processing unit may specify the interface by performing processing for comparing an amplitude value in an A mode waveform that is based on the reception signal or a luminance value in a B mode image that is based on the reception signal with a predetermined threshold value.

According to this configuration, the processing unit can specify the interface between the test subject and the ultrasonic measurement sheet based on an A mode waveform or a B mode image resulting from ultrasonic echoes from the interface, thus making it possible to acquire depth information regarding the interface in ultrasonic measurement.

Also, in the first aspect of the invention, the processing unit may calculate a luminance variance for a plurality of depths in a B mode image that is based on the reception signal, and specify the interface between the ultrasonic measurement sheet and the test subject based on the calculated luminance variances.

According to this configuration, the processing unit can specify the interface between the test subject and the ultrasonic measurement sheet based on a B mode image luminance variance, thus making it possible to acquire depth information regarding the interface in ultrasonic measurement even if ultrasonic echoes from the interface are weak.

Also, in the first aspect of the invention, the processing unit may perform processing for analyzing, based on the reception signal, code information that is recorded in the ultrasonic measurement sheet, and acquire the reference thickness information of the ultrasonic measurement sheet based on the code information.

According to this configuration, the processing unit can perform code information analysis processing and acquire appropriate reference thickness information that corresponds to the ultrasonic measurement sheet that is to be used, thus making it possible to acquire inclination information regarding the ultrasonic probe using appropriate reference thickness information.

Also, in the first aspect of the invention, the processing unit may perform processing for acquiring, based on the reception signal, code information that is recorded in the ultrasonic measurement sheet, and specify a scan location of the ultrasonic probe with respect to the ultrasonic measurement sheet based on the acquired code information, and the processing unit may acquire the inclination information regarding the ultrasonic probe at the specified scan location based on the reception signal.

According to this configuration, the processing unit can specify the scan location of the ultrasonic probe with respect to the ultrasonic measurement sheet, and acquire inclination information regarding the ultrasonic probe at the specified scan location. As a result, even a user who is not familiar with operating the ultrasonic measuring device can hold the ultrasonic probe at the appropriate scan location and so as to not be inclined while performing ultrasonic measurement.

Also, in the first aspect of the invention, the ultrasonic measurement sheet may have an ultrasound transmissive medium and a plurality of reflectors embedded in the ultrasound transmissive medium, the code information may be recorded using at least one of the reflectance, the number, the shape, and the size of the plurality of reflectors, the reception unit may perform processing for receiving an ultrasonic echo from the plurality of reflectors, and output a reception signal to the processing unit, and the processing unit may perform processing for analyzing the code information based on the reception signal from the reception unit.

According to this configuration, the processing unit can analyze code information that was recorded using at least one of the reflectance, the number, the shape, and the size of the reflectors, and acquire the reference thickness information of the ultrasonic measurement sheet or specify the scan location of the ultrasonic probe.

Also, in the first aspect of the invention, the ultrasonic measurement sheet may have an ultrasound transmissive medium and a plurality of reflectors arranged in an array in the ultrasound transmissive medium, the reception unit may perform processing for receiving an ultrasonic echo from the plurality of reflectors, and output a reception signal to the processing unit, and the processing unit may analyze the number of reflections by the plurality of reflectors based on the reception signal, and acquire the inclination information regarding the ultrasonic probe based on the number of reflections.

According to this configuration, the processing unit can acquire inclination information regarding the ultrasonic probe using an ultrasonic measurement sheet that has multiple reflectors by analyzing the number of reflections by the reflectors.

Also, in the first aspect of the invention, in a case of determining based on the acquired inclination information that the ultrasonic probe is inclined, the processing unit may generate and output notification data for notification of the fact that the ultrasonic probe is inclined.

According to this configuration, the user can become aware of whether or not the ultrasonic probe is inclined, thus making it possible for the ultrasonic probe to be held so as to not be inclined relative to the test subject during measurement. As a result, it is possible to, for example, accurately measure the fat layer thickness, muscle layer thickness, or the like of the test subject.

Also, another aspect of the invention relates to an ultrasonic image device including: any of the above-described ultrasonic measuring devices; and a display unit that displays display image data.

### BRIEF DESCRIPTION OF THE DRAWINGS

FIG. 1 shows an example of basic configurations of an ultrasonic measuring device and an ultrasonic image device.

FIGS. 2A and 2B show an example of a basic configuration of an ultrasonic probe.

FIGS. 3A and 3B are diagrams for describing the acquisition of depth information in a first configuration example.

FIGS. 4A and 4B are diagrams for describing the acquisition of depth information in the first configuration example in the case where the ultrasonic probe is inclined.

FIG. 5 is a diagram for describing the detection of the angle of inclination of the ultrasonic probe.

FIG. 6 is an example of a flowchart of processing for acquiring inclination information regarding the ultrasonic probe in the first configuration example.

FIGS. 7A and 7B are diagrams for describing the acquisition of depth information in a second configuration example.

FIG. 8 is an example of a flowchart of processing for acquiring inclination information regarding the ultrasonic probe in the second configuration example.

FIGS. 9A and 9B show an example of a configuration of an ultrasonic measurement sheet used in a third configuration example.

FIGS. 10A and 10B are diagrams for describing the acquisition of inclination information regarding the ultrasonic probe in the third configuration example.

FIG. 11 is a diagram for describing the number of reflections by reflectors.

FIG. 12 shows examples of ultrasonic images resulting from ultrasonic echoes from reflectors.

FIG. 13 is an example of a flowchart of processing for acquiring inclination information regarding the ultrasonic probe in the third configuration example.

FIGS. 14A and 14B show an example of a configuration of an ultrasonic measurement sheet 200 used in a fourth configuration example.

FIG. 15A shows an example of code information.

FIG. 15B shows an example of a luminance table.

FIG. 16 shows an example of ultrasonic images of reflector groups.

FIG. 17 is an example of a flowchart of code information analysis processing in the fourth configuration example.

FIG. 18 shows an example of a configuration of an ultrasonic measurement sheet used in a fifth configuration example.

FIGS. 19A and 19B show examples of specific configurations of an ultrasonic image device. FIG. 19C shows an example of a specific configuration of an ultrasonic probe.

### DESCRIPTION OF EXEMPLARY EMBODIMENTS

The following is a detailed description of preferred embodiments of the invention. Note that the embodiments described below are not intended to unduly limit the content of the invention recited in the claims, and all of the configurations described in the embodiments are not necessarily essential as solutions provided by the invention.

1. Ultrasonic Measuring Device

1-(1) Basic Configuration Example

FIG. 1 shows an example of the basic configurations of an ultrasonic measuring device 100 and an ultrasonic image device 400 of this embodiment. The ultrasonic measuring device 100 of this embodiment includes an emission unit 110, a reception unit 120, and a processing unit 130. Also, the ultrasonic image device 400 of this embodiment includes the ultrasonic measuring device 100 and a display unit 410 (in a broad sense, a notification unit 420). Note that the ultrasonic measuring device 100 and the ultrasonic image device 400 of this embodiment are not limited to the configurations shown in FIG. 1, and various modifications can be carried out, such as omitting some of the constituent elements, replacing some of the constituent elements with other constituent elements, and adding other constituent elements.

The emission unit 110 performs ultrasound emission processing. Specifically, the emission unit 110 outputs an emission signal (drive signal), which is an electrical signal, to an ultrasonic probe 300, and the ultrasonic probe 300 emits ultrasound toward a subject via an ultrasonic measurement sheet 200. The ultrasonic probe 300 includes an ultrasonic transducer device (not shown), and the ultrasonic transducer device converts the emission signal, which is an electrical signal, into ultrasound.

The reception unit 120 performs ultrasonic echo reception processing. Specifically, the ultrasonic transducer device included in the ultrasonic probe 300 converts ultrasonic echoes from the subject (test subject) and the ultrasonic measurement sheet 200 into an electrical signal. The reception unit 120 performs reception processing such as amplification, wave detection, A/D conversion, and phase matching on a reception signal (analog signal), which is an electrical signal, from the ultrasonic transducer device, and outputs the reception signal (digital data) that is the result of the reception processing to the processing unit 130.

The processing unit 130 performs ultrasonic measurement control processing. Specifically, the processing unit 130 performs processing for controlling the emission unit 110 and the reception unit 120, and processing for generating ultrasonic image data based on a reception signal from the reception unit 120. The processing unit 130 can be realized with an FPGA (Field-Programmable Gate Array), for example.

Also, the processing unit 130 acquires inclination information regarding the ultrasonic probe 300 based on a reception signal resulting from ultrasonic echoes from the interface between the test subject and the ultrasonic measurement sheet 200, or resulting from ultrasonic echoes from the ultrasonic measurement sheet 200. Specifically, based on the reception signal, the processing unit 130 acquires depth information regarding the interface between the test subject and the ultrasonic measurement sheet 200 in ultrasonic measurement, and then acquires the inclination information by performing processing for comparing the depth information with reference thickness information of the ultrasonic measurement sheet 200.

The depth information is information regarding the depth (deepness) in the ultrasound emission direction from the face of the ultrasonic measurement sheet 200 that opposes the ultrasonic probe 300 to the interface between the ultrasonic measurement sheet 200 and the test subject. Specifically, it is pixel values in the depth direction at positions corresponding to the interface in a B mode image obtained by ultrasonic measurement, for example.

The inclination information is information indicating whether or not the axial direction of the ultrasonic probe 300 is inclined relative to the normal direction of the surface of the test subject. Alternatively, it is information indicating whether or not the axial direction of the ultrasonic probe 300 is inclined relative to the normal direction of the interface between the ultrasonic measurement sheet 200 and the test subject. The axial direction of the ultrasonic probe 300 is a direction perpendicular to the scan direction and the slice direction. The state in which the ultrasonic probe 300 is inclined refers to a state in which the axial direction of the ultrasonic probe 300 is not parallel to the normal direction of the surface of the test subject (or the aforementioned interface). The state in which the ultrasonic probe 300 is not inclined refers to a state in which the axial direction of the ultrasonic probe 300 is parallel to the normal direction of the surface of the test subject (or the aforementioned interface).

Alternatively, the inclination information is an angle 8 obtained using the equation θ=90°−φ, where φ is the angle formed by the surface of the test subject (or the aforementioned interface) and the scanning plane of the ultrasonic probe 300. The state in which the ultrasonic probe 300 is inclined refers to a state in which θ>0°, and the state in which the ultrasonic probe 300 is not inclined refers to a state in which θ=0°.

The reference thickness information of the ultrasonic measurement sheet 200 is information regarding the thickness (length in the depth direction) of the ultrasonic measurement sheet 200, and is information indicating the thickness used as a reference for acquiring inclination information regarding the ultrasonic probe 300 based on the interface depth information in the processing for acquiring inclination information performed by the processing unit 130.

In the case of determining that the ultrasonic probe 300 is inclined, the processing unit 130 generates and outputs notification data for notification of the fact that the ultrasonic probe 300 is inclined. The depth information acquisition and the inclination information determination performed by the processing unit 130 will be described in detail later.

The notification data may include display image data to be displayed by the display unit 410, audio data to be output from a speaker, or the like. Specifically, the notification data is data representing an image (illustration) displaying the fact that the ultrasonic probe 300 is inclined, or data representing an audio message for informing the user that the ultrasonic probe 300 is inclined. Alternatively, the notification data is data regarding a control signal for changing the pitch or volume of a beep sound, a control signal for lighting or blinking an LED, a control signal for causing a vibrator to vibrate, or the like.

Also, in the case of performing ultrasonic measurement using an ultrasonic measurement sheet 200 that has multiple reflectors, the processing unit 130 can also analyze the number of reflections by the reflectors based on the reception signal, and acquire inclination information regarding the ultrasonic probe 300 based on the number of reflections. The inclination information determination performed based on the number of reflections will be described in detail later.

The notification unit 420 is, for example, the display unit 410, which is a liquid crystal display or the like, or a speaker (not shown), and is for notifying the user of the notification data from the processing unit 130. The display unit 410 is a display device such as a liquid crystal display or an organic EL display, and is for displaying display image data from the processing unit 130. The notification unit 420 may be a beeper that generates a beep sound, an LED that is lit or blinked, a vibrator that vibrates, or the like.

The ultrasonic measurement sheet 200 is a sheet provided between the ultrasonic probe 300 and the test subject in order to ensure acoustic matching (acoustic impedance matching) between the ultrasonic probe 300 and the test subject during ultrasonic measurement. The ultrasonic measurement sheet 200 is constituted by an ultrasound transmissive medium that has an acoustic impedance close to that of the test subject and transmits ultrasound, and is affixed to the surface of the test subject when ultrasonic measurement is to be performed.

Also, the ultrasonic measurement sheet 200 may include multiple reflectors that are formed from a material that has a different acoustic impedance from the ultrasound transmissive medium. The processing unit 130 can acquire inclination information regarding the ultrasonic probe 300 based on a reception signal resulting from ultrasonic echoes from the reflectors. Alternatively, the processing unit 130 can acquire the reference thickness information of the ultrasonic measurement sheet 200 or scan location information of the ultrasonic probe 300 by analyzing code information recorded in the ultrasonic measurement sheet 200. The reflectors and the code information of the ultrasonic measurement sheet 200 will be described later.

According to the ultrasonic measuring device 100 of this embodiment, the processing unit 130 can acquire inclination information regarding the ultrasonic probe 300 based on a reception signal resulting from ultrasonic echoes from the interface between the test subject and the ultrasonic measurement sheet 200 or resulting from ultrasonic echoes from the ultrasonic measurement sheet 200. Also, upon determining that the ultrasonic probe 300 is inclined, the processing unit 130 can generate and output notification data for notification of the fact that the ultrasonic probe 300 is inclined. According to this configuration, the user can become aware of whether or not the ultrasonic probe 300 is inclined, thus making it possible for the ultrasonic probe 300 to be held perpendicular to the surface of the test subject during measurement. As a result, it is possible to, for example, accurately measure the fat layer thickness, muscle layer thickness, or the like of the test subject.

FIGS. 2A and 2B show an example of the basic configuration of the ultrasonic probe 300 used along with the ultrasonic measuring device 100 of this embodiment. The ultrasonic probe 300 includes an ultrasonic transducer device 310. Note that the ultrasonic probe 300 of this embodiment is not limited to the configuration shown in FIGS. 2A and 2B, and various modifications can be carried out, such as omitting some of the constituent elements, replacing some of the constituent elements with other constituent elements, and adding other constituent elements.

As shown in FIGS. 2A and 2B, the scan direction is the X direction, the slice direction is the Y direction, and the depth direction is the Z direction. FIG. 2A is a view of the ultrasonic probe 300 along the Y direction (front view), and FIG. 2B is a view of the ultrasonic probe 300 along the X direction (side view).

The ultrasonic transducer device 310 includes multiple ultrasonic transducer elements (not shown). The ultrasonic transducer device 310 converts an emission signal, which is an electrical signal, into ultrasound, and converts ultrasonic echoes from the subject (test subject) and the ultrasonic measurement sheet 200 into an electrical signal. The ultrasonic transducer elements may be thin-film piezoelectric ultrasonic transducer elements, bulk piezoelectric ultrasonic transducer elements, CMUT (Capacitive Micromachined Ultrasonic Transducer) elements, or the like.

1-(2) First Configuration Example

FIGS. 3A and 3B are diagrams for describing the acquisition of depth information in a first configuration example of the ultrasonic measuring device 100 of this embodiment. In the first configuration example, the processing unit 130 specifies an interface BS between the test subject and the ultrasonic measurement sheet 200 by comparing an amplitude value in an A mode waveform that is based on the reception signal or a luminance value in a B mode image that is based on the reception signal with a predetermined threshold value.

As shown in FIG. 3A, if the ultrasonic probe 300 is not inclined, that is to say, if the angle formed by the scanning plane of the ultrasonic probe 300 and the plane (X-Z plane) that is orthogonal to the interface BS between the test subject and the ultrasonic measurement sheet 200 is 0°, the depth in the ultrasound emission direction from the face of the ultrasonic measurement sheet 200 that opposes the ultrasonic probe to the interface BS is the same as a thickness DA of the ultrasonic measurement sheet 200.

FIG. 3B shows an example of an ultrasonic image (B mode image) in the case where the ultrasonic probe 300 is not inclined. As shown in FIG. 3B, the portion of the B mode image that corresponds to the interface BS has a higher luminance due to the reflected waves from the interface BS. By comparing the luminance value of the B mode image with a predetermined threshold value, the processing unit 130 can specify the depth of the interface BS and acquire depth information regarding the interface BS. Specifically, the processing unit 130 acquires a depth direction coordinate value bz of the high luminance portion that corresponds to the interface BS as the depth information. The depth direction coordinate value bz can be expressed in units of image pixels. In the case of FIG. 3B, the depth direction coordinate value bz of the portion that corresponds to the interface BS is a pixel value da that corresponds to the thickness DA of the ultrasonic measurement sheet 200. Note that in the following description, the depth direction coordinate value (pixel value) bz is sometimes called the depth bz.

FIGS. 4A and 4B are diagrams for describing the acquisition of depth information in the first configuration example of the ultrasonic measuring device 100 of this embodiment, in the case where the ultrasonic probe 300 is inclined.

As shown in FIG. 4A, if the ultrasonic probe 300 is inclined, the depth DX in the ultrasound emission direction from the face of the ultrasonic measurement sheet 200 that opposes the ultrasonic probe to the interface BS is greater than the thickness DA of the ultrasonic measurement sheet 200. As shown in FIG. 4B, the depth bz obtained by the processing unit 130 by comparing the luminance value of the B mode image with a predetermined threshold value is a depth dx that is greater than the depth da that corresponds to the thickness DA of the ultrasonic measurement sheet 200. Accordingly, by comparing the depth dx (in a broad sense, the depth information) of the interface BS obtained from the B mode image with the depth da that corresponds to the thickness DA of the ultrasonic measurement sheet 200, the processing unit 130 can determine whether or not the ultrasonic probe 300 is inclined.

FIG. 5 is a diagram for describing the detection of the angle of inclination of the ultrasonic probe 300 in the first configuration example of the ultrasonic measuring device 100 of this embodiment. As shown in FIG. 5, the angle of inclination θ of the ultrasonic probe 300 can be obtained with the following equation using the depth DX in the ultrasound emission direction from the face of the ultrasonic measurement sheet 200 that opposes the ultrasonic probe to the interface BS and the thickness DA of the ultrasonic measurement sheet 200.

θ=arccos(DA/DX) (1)

Accordingly, the processing unit 130 can calculate the angle of inclination θ of the ultrasonic probe 300 using Equation 1, based on the depth dx of the interface BS obtained from the B mode image and the depth da that corresponds to the thickness DA of the ultrasonic measurement sheet 200.

FIG. 6 is an example of a flowchart of processing for acquiring inclination information regarding the ultrasonic probe 300 in the first configuration example of the ultrasonic measuring device 100 of this embodiment. The processing flow shown in FIG. 6 is executed by the processing unit 130.

Firstly, the processing unit 130 sets the scan direction coordinate value at the center of the B mode image as the scan direction coordinate value bx of the pixel that is to be subjected to the B mode image luminance value comparison (step S11). Specifically, the scan direction coordinate value bx can be expressed in units of image pixels. Note that the coordinate value bx of the pixel that is to be subjected to the luminance value comparison does not need to be the scan direction coordinate value at the center of the image.

Next, the processing unit 130 sets the depth direction coordinate value bz (depth bz) of the pixel that is to be subjected to the luminance value comparison to “0” (step S12).

Next, the processing unit 130 compares the luminance value L(bx,bz) of the pixel that corresponds to the scan direction coordinate value bx and the depth bz with a predetermined threshold value Lth (step S13). The predetermined threshold value Lth is a value that is, for example, less than the luminance value of a pixel that corresponds to the interface BS, and greater than the luminance value of a pixel that corresponds to the ultrasonic measurement sheet 200.

If the luminance value L(bx,bz) is greater than the predetermined threshold value Lth, the processing unit 130 determines that that pixel corresponds to the interface BS (step S14). In other words, the processing unit 130 determines that the depth bz of that pixel is the depth of the interface BS.

On the other hand, if the luminance value L(bx,bz) is less than or equal to the predetermined threshold value Lth, the processing unit 130 increments the depth bz, that is to say bz=bz+1 (step S15). The procedure then returns to step S13, and the luminance value comparison is repeated. In this way, the processing unit 130 repeats steps S13 and S15 while incrementing the depth bz until the luminance value L(bx,bz) is greater than the threshold value Lth.

In step S16 that follows step S14, it is determined whether or not the absolute value abs(bz−da), which is the absolute value of the difference between the depth bz of the interface BS and the depth da that corresponds to the thickness DA of the ultrasonic measurement sheet 200, is less than or equal to an allowable error value err. This allowable error value err is a value that is, for example, greater than a value that corresponds to variation in the thickness DA of the ultrasonic measurement sheet 200, and less than an allowable value for accurately determining whether or not the ultrasonic probe 300 is inclined.

If the absolute value abs(bz−da) of the difference is less than or equal to the allowable error value err, the processing unit 130 determines that the ultrasonic probe 300 is not inclined (step S17).

If the absolute value abs(bz−da) of the difference is greater than the allowable error value err, the processing unit 130 determines that the ultrasonic probe 300 is inclined (step S18). The processing unit 130 then generates notification data for notification of the fact that the ultrasonic probe 300 is inclined, and outputs the notification data to the notification unit 420 (step S19).

In the processing flow shown in FIG. 6, the processing unit 130 specifies the interface BS based on the luminance values in the B mode image, but the processing unit 130 may specify the interface BS based on the amplitude values in an A mode waveform. Specifically, in this case, the processing unit 130 compares the amplitude value of the reception signal with a predetermined threshold value, and detects the time period (delay time period) from when ultrasound is emitted until when the amplitude value exceeds the predetermined threshold value. The processing unit 130 can then specify the interface BS based on this delay time period. In other words, it can acquire the depth (in a broad sense, the depth information) of the interface BS.

In this way, according to the first configuration example of the ultrasonic measuring device 100 of this embodiment, the processing unit 130 can specify the interface BS between the ultrasonic measurement sheet 200 and the test subject by comparing the amplitude values of an A mode waveform or the luminance values of a B mode image with a predetermined threshold value. The processing unit 130 can then acquire inclination information regarding the ultrasonic probe 300 by performing processing for comparing the depth information of the interface BS with reference thickness information of the ultrasonic measurement sheet 200.

1-(3) Second Configuration Example

FIGS. 7A and 7B are diagrams for describing the acquisition of depth information in a second configuration example of the ultrasonic measuring device 100 of this embodiment. In the second configuration example, the processing unit 130 can calculate a luminance variance for various depths in the B mode image that is based on the reception signal, and specify the interface BS between the ultrasonic measurement sheet 200 and the test subject based on the calculated luminance variances.

If the acoustic impedances of the ultrasonic measurement sheet 200 and the test subject are the same or substantially the same, there are cases where the reflection of ultrasound at the interface BS is weak, and the portion of the B mode image that corresponds to the interface BS does not have a high luminance. In such cases, it is difficult to specify the interface BS with the ultrasonic measuring device 100 of the first configuration example described above, but the interface BS can be specified based on the luminance variance with the ultrasonic measuring device 100 of the second configuration example.

FIG. 7A shows an example of an ultrasonic image (B mode image) in the case where the acoustic impedances of the ultrasonic measurement sheet 200 and the test subject are the same or substantially the same, that is to say, the case where ultrasound is not reflected at the interface BS.

As shown in FIG. 7A, the portion corresponding to the interface BS does not have a high luminance since ultrasound is not reflected at the interface BS. Also, there are almost no reflected waves from the interior of the ultrasonic measurement sheet 200 since the ultrasonic measurement sheet 200 is formed by an ultrasound transmissive medium that has a uniform acoustic impedance. Accordingly, the portion of the B mode image that corresponds to the ultrasonic measurement sheet 200 has a low and uniform luminance. On the other hand, the test subject (e.g., a human body) contains various organs and does not have a uniform acoustic impedance. The portion of the B mode image that corresponds to the test subject therefore does not have a uniform luminance. In view of this, the processing unit 130 can calculate the luminance variance for various depths in the B mode image and specify the interface BS between the ultrasonic measurement sheet 200 and the test subject based on the calculated luminance variances.

FIG. 7B is a diagram for describing the calculation of the luminance variance for various depths by the processing unit 130. As shown in FIG. 7B, the processing unit 130 obtains a luminance variance Lvar(0) for N+1 (N being a natural number) pixels in the B mode image for which scan direction coordinate value bx=0 to N and depth bz=0. Next, a luminance variance Lvar(1) is obtained for N+1 pixels for which bx=0 to N and bz=1. The processing unit 130 then obtains luminance variances Lvar(2), Lvar(3), Lvar(4), and so on for pixels for which bz=2, 3, 4, and so on.

The luminance variance Lvar(bz) is obtained with the following equation.

$$Lvar ( bz ) = \frac{1}{( N + 1 )} {\sum }_{bx = 0}^{N} {( L ( bx , bz ) - AL ( bz ) )}^{2} \qquad ( 2 )$$

Here, AL(bz) is the average luminance value for the N+1 pixels having the depth bz, and is obtained with the following equation.

$$AL ( bz ) = \frac{1}{( N + 1 )} {\sum }_{bx = 0}^{N} L ( bx , bz ) \qquad ( 3 )$$

The portion that corresponds to the ultrasonic measurement sheet 200 has a uniform luminance, and therefore has a small luminance variance. On the other hand, the portion that corresponds to the test subject does not have a uniform luminance, and therefore has a higher luminance variance than the portion corresponding to the ultrasonic measurement sheet 200 does. Accordingly, the processing unit 130 can specify the interface BS between the ultrasonic measurement sheet 200 and the test subject based on the luminance variance for various depths bz. Specifically, if the luminance variance at a certain depth bz is greater than a predetermined threshold value, the processing unit 130 can determine that that depth bz is the depth of the interface BS.

FIG. 8 is an example of a flowchart of processing for acquiring inclination information regarding the ultrasonic probe 300 in the second configuration example of the ultrasonic measuring device 100 of this embodiment. The processing flow shown in FIG. 8 is executed by the processing unit 130.

Firstly, the processing unit 130 sets the depth bz of the pixel to be used in the luminance variance calculation to “0” (step S21). Next, the processing unit 130 calculates the luminance variance Lvar(bz) for the N+1 pixels for which bx=0 to N (step S22).

Next, the processing unit 130 determines whether or not the luminance variance Lvar(bz) is greater than a predetermined threshold value Lvar_th (step S23). The predetermined threshold value Lvar_th is a value that is, for example, greater than the luminance variance of a portion of the B mode image that corresponds to the ultrasonic measurement sheet 200, and less than the luminance variance of a portion that corresponds to the test subject.

If the luminance variance Lvar(bz) is greater than the predetermined threshold value Lvar_th, the processing unit 130 determines that that depth bz is the depth of the interface BS (step S24).

On the other hand, if the luminance variance Lvar(bz) is less than or equal to the predetermined threshold value Lvar_th, the processing unit 130 increments the depth bz, that is to say bz=bz+1 (step S25). Steps S22 and S23 are then repeated. In this way, the processing unit 130 repeats steps step S22, S23, and S25 while incrementing the depth bz until the luminance variance Lvar(bz) is greater than the predetermined threshold value Lvar_th.

In step S26 that follows step S24, it is determined whether or not the absolute value abs(bz−da), which is the absolute value of the difference between the depth bz of the interface BS and the depth da that corresponds to the thickness DA of the ultrasonic measurement sheet 200, is less than or equal to an allowable error value err.

Steps S26 to S29 will not be described in detail here since they are same as in the above-described processing flow (FIG. 6) of the first configuration example.

In this way, according to the second configuration example of the ultrasonic measuring device 100 of this embodiment, the processing unit 130 can calculate a luminance variance for various depths in the B mode image and specify the interface BS between the ultrasonic measurement sheet 200 and the test subject based on the calculated luminance variances. The processing unit 130 can then acquire inclination information regarding the ultrasonic probe 300 by performing processing for comparing the depth information of the interface BS with reference thickness information of the ultrasonic measurement sheet 200.

1-(4) Third Configuration Example

FIGS. 9A and 9B show an example of a configuration of the ultrasonic measurement sheet 200 used in a third configuration example of the ultrasonic measuring device 100 of this embodiment. The ultrasonic measurement sheet 200 includes an ultrasound transmissive medium 210 and multiple reflectors 222. FIG. 9A is a top view of the ultrasonic measurement sheet 200, and FIG. 9B is a cross-sectional view of the ultrasonic measurement sheet 200. The direction parallel to one of the sides of the ultrasonic measurement sheet 200 is the X direction, and the direction that is perpendicular to the X direction and parallel to the sheet surface is the Y direction. Also, the direction that is perpendicular to the sheet surface, that is to say the thickness direction of the sheet, is the Z direction.

It is desirable that the ultrasound transmissive medium 210 is formed from a material that transmits ultrasound, has an acoustic impedance close to that of the test subject, and has little attenuation. For example, it can be formed from an oil gel, acrylamide, a hydro gel, or the like. This ultrasound transmissive medium 210 is used in close contact with the test subject.

The reflectors 220 each have a narrow columnar shape, are formed from a material that has a different acoustic impedance from the ultrasound transmissive medium 210, and are provided in the ultrasound transmissive medium 210 such that their lengthwise direction conforms to the Z direction. The reflectors are arranged at even intervals in an array in the X-Y plane. Due to having a different acoustic impedance from the ultrasound transmissive medium 210, the reflectors 222 reflect some of the ultrasound that enters the ultrasonic measurement sheet 200. Rubber or the like can be used as the material for the reflectors 222. Note that the reflectors 220 are not limited to having a narrow columnar shape, and may have a narrow prismatic shape.

FIGS. 10A and 10B are diagrams for describing the acquisition of inclination information regarding the ultrasonic probe 300 in the third configuration example of the ultrasonic measuring device 100 of this embodiment. FIG. 10A shows the case where the ultrasonic probe 300 is not inclined, and FIG. 10B shows the case where the ultrasonic probe 300 is inclined.

As shown in FIG. 10A, if the ultrasonic probe 300 is not inclined, the ultrasound emitted from the ultrasonic probe 300 passes through the ultrasonic measurement sheet 200 without being reflected by any of the reflectors 222, or a portion of the ultrasound is reflected by any one of the reflectors 222.

On the other hand, as shown in FIG. 10B, if the ultrasonic probe 300 is inclined, a portion of the ultrasound emitted from the ultrasonic probe 300 is reflected by two or more reflectors among the reflectors 222. Accordingly, the processing unit 130 can analyze the number of reflections by the reflectors 222 based on the reception signal resulting from ultrasonic echoes from the reflectors 222, and acquire inclination information regarding the ultrasonic probe 300 based on the number of reflections.

FIG. 11 is a diagram for describing the number of reflections by the reflectors 222. LA is the lengthwise direction length of each of the reflectors 222, and SA is the space between reflectors. The following describes the number of reflections with respect to the ultrasound emission directions (scanning planes) indicated by B1 to B5 in FIG. 11.

If the ultrasonic probe 300 is not inclined, the scanning plane is as shown by B1 or B2 in FIG. 11. In the case of B1 in FIG. 11, the ultrasound passes through without being reflected by a reflector 222, and therefore the number of reflections is “0”. Also, in the case of B2 in FIG. 11, the ultrasound is reflected at one end and the other end of one reflector 222, and therefore the number of reflections is “2”.

If the ultrasonic probe 300 is inclined, the scanning plane is as shown by B3, B4, or B5 in FIG. 11, for example. In the case of B3 in FIG. 11, the ultrasound is reflected at one end (the ultrasonic probe side) of one reflector 222 and at the other end (test subject side) of an adjacent reflector 222, and therefore the number of reflections is “2”. In the case of B4 in FIG. 11, in which the ultrasonic probe 300 is more inclined, the ultrasound is reflected two times by one reflector 222, and reflected one time by an adjacent reflector 222, and therefore the number of reflections is “3”. In the case of B5 in FIG. 11, in which the ultrasonic probe 300 is even more inclined, the ultrasound is reflected one time by one reflector 222, reflected two times by an adjacent reflector 222, and reflected one time by another adjacent reflector 222, and therefore the number of reflections is “4”. Although not shown, if the ultrasonic probe 300 is even more inclined, the number of reflections will be “5” or more.

In this way, the number of reflections increases the more the ultrasonic probe 300 is inclined. Accordingly, the processing unit 130 can determine whether or not the ultrasonic probe 300 is inclined by analyzing the number of reflections based on the reception signal resulting from ultrasonic echoes from the reflectors 222. Specifically, as shown in FIG. 11, if the number of reflections is “3” or more, the processing unit 130 can determine that the ultrasonic probe 300 is inclined.

The number of reflections is “2” in both of the cases of B2 and B3 in FIG. 11, and it is not possible to distinguish between them. However, if the length LA of the reflectors 222 is set sufficiently greater than the space SA (e.g., LA≧10×SA), the inclination of the ultrasonic probe 300 in the case of B3 in FIG. 11 will be small enough to be ignored, and thus there will be no practical problem.

FIG. 12 shows examples of ultrasonic images (B mode images) resulting from ultrasonic echoes from the reflectors 222 in the third configuration example of the ultrasonic measuring device 100 of this embodiment. FIG. 12 shows examples of B mode images in the case where the number of reflections is “0” (the case of B1 in FIG. 11), the case where the number of reflections is “2” (the cases of B2 and B3 in FIG. 11), the case where the number of reflections is “3” (the case of B4 in FIG. 11), and the case where the number of reflections is “4” (the case of B5 in FIG. 11).

As shown in FIG. 12, with the third configuration example of the ultrasonic measuring device 100, the number of reflections by the reflectors 222 can be analyzed by counting, along the depth direction, the number of portions of the B mode image in which the luminance is higher than a predetermined threshold value.

FIG. 13 is an example of a flowchart of processing for acquiring inclination information regarding the ultrasonic probe 300 in the third configuration example of the ultrasonic measuring device 100 of this embodiment. The processing flow shown in FIG. 13 is executed by the processing unit 130.

Firstly, the processing unit 130 sets the scan direction coordinate value bx of the pixel that is to be subjected to the B mode image luminance value comparison to “0” (step S31).

Next, the processing unit 130 sets the depth bz of the pixel that is to be subjected to the luminance value comparison to “0”, and sets a number of reflections count value n to “0” (step S32).

Next, the processing unit 130 compares the luminance value L(bx,bz) of the pixel that corresponds to the scan direction coordinate value bx and the depth bz with the predetermined threshold value Lth (step S33). The predetermined threshold value Lth is a value that is, for example, less than the luminance value of a pixel that corresponds to a reflector 222, and greater than the luminance value of a pixel that corresponds to a portion of the ultrasonic measurement sheet 200 that does not correspond to a reflector.

If the luminance value L(bx,bz) is greater than the predetermined threshold value Lth, the processing unit 130 increments the number of reflections count value n (step S34). On the other hand, if the luminance value L(bx,bz) is less than or equal to the predetermined threshold value Lth, the processing unit 130 does not increment the number of reflections count value n.

Next, the processing unit 130 increments the depth bz, that is to say bz=bz+1 (step S35), and determines whether or not the depth bz is greater than the depth da that corresponds to the thickness DA of the ultrasonic measurement sheet 200 (step S36).

If the depth bz is less than or equal to da, the processing unit 130 returns to step S33 and compares the luminance value L(bx,bz) with the predetermined threshold value Lth. Then, if the luminance value L(bx,bz) is greater than the predetermined threshold value Lth, the processing unit 130 increments the number of reflections count value n (step S34). In this way, the processing unit 130 counts the number of reflections until the depth bz is greater than the depth da that corresponds to the thickness DA of the ultrasonic measurement sheet 200.

If the depth bz is greater than da, the processing unit 130 determines whether or not the number of reflections count value n is greater than or equal to “3” (step S37). If the number of reflections count value n is greater than or equal to “3”, the processing unit 130 determines that the ultrasonic probe 300 is inclined (step S38). The processing unit 130 then generates notification data for notification of the fact that the ultrasonic probe 300 is inclined, and outputs the notification data to the notification unit 420 (step S39).

On the other hand, if the number of reflections count value n is less than “3”, the processing unit 130 increments the scan direction coordinate value bx, that is to say bx=bx+1 (step S40), and determines whether or not the coordinate value bx is lower than a scan width W (step S41). The scan width W is a value that corresponds to the number of pixels along the scan direction in the B mode image, for example.

If the coordinate value bx is lower than the scan width W, the processing unit 130 returns to step S32 and repeats the number of reflections count processing in steps S32 to S37. In this way, the processing unit 130 repeats the number of reflections count processing until the number of reflections count value n is greater than or equal to “3”, or until the scan direction coordinate value bx is greater than or equal to the scan width W in the case where the number of reflections count value n is not greater than or equal to “3”.

If the scan direction coordinate value bx exceeds the scan width W while the number of reflections count value n is not greater than or equal to “3”, the processing unit 130 determines that the ultrasonic probe 300 is not inclined (step S42).

In the processing flow shown in FIG. 13, the processing unit 130 counts the number of reflections based on the luminance values of a B mode image, but the processing unit 130 may count the number of reflections based on the amplitude values of an A mode waveform. Specifically, in this case, the processing unit 130 can compare the amplitude value of the reception signal with a predetermined threshold value in a predetermined delay time period from when ultrasound is emitted, and count the number of reflections by counting the number of signal pulses having an amplitude value that is greater than the predetermined threshold value.

In this way, according to the third configuration example of the ultrasonic measuring device 100 of this embodiment, the processing unit 130 can analyze the number of reflections by the reflectors 222 of the ultrasonic measurement sheet 200 based on the reception signal, and acquire inclination information regarding the ultrasonic probe 300 based on the number of reflections.

1-(5) Fourth Configuration Example

FIGS. 14A and 14B show an example of a configuration of an ultrasonic measurement sheet 200 used in a fourth configuration example of the ultrasonic measuring device 100 of this embodiment. FIG. 14A is a top view of the ultrasonic measurement sheet 200, and FIG. 14B is a cross-sectional view of the ultrasonic measurement sheet 200. The ultrasonic measurement sheet 200 includes an ultrasound transmissive medium 210 and multiple reflectors 220 (220-1 to 220-4).

The ultrasonic measurement sheet 200 shown in FIGS. 14A and 14B can record code information using the reflectors 220. Since the ultrasound transmissive medium 210 has been described with reference to FIG. 9, it will not be described in detail here.

The reflectors 220 are formed from a material that has a different acoustic impedance from the ultrasound transmissive medium 210, and they are embedded in the ultrasound transmissive medium 210. The reflectors 220 reflect ultrasound due to having a different acoustic impedance from the ultrasound transmissive medium 210. Rubber or the like can be used as the material for the reflectors 220. The code information is recorded using at least one of the reflectance, the number, the shape, and the size of the reflectors 220. Specifically, the code information is recorded by setting at least one of the reflectance, the number, the shape, and the size to a predetermined value. For example, the code information can be recorded by setting the reflectances of the reflectors 220 to any of multiple predetermined reflectances.

Letting Z1 be the acoustic impedance of the ultrasound transmissive medium 210, and Z2 be the acoustic impedance of a reflector 220, the reflectance R of that reflector 220 is obtained with the following equation.

R=(Z2−Z1)/(Z1+Z2) (4)

Also, the acoustic impedance Z is obtained with the following equation.

Z=ρ×c (5)

Here, ρ is the density of the medium, and c is the acoustic velocity in the medium.

Accordingly, the acoustic impedance Z2 can be set variably by changing the material used for the reflectors 220. For example, it is possible to use silicone-based rubber or the like as the base material for the reflector 220, and change the acoustic impedance Z2 of the reflector 220 by mixing in a filler such as a metal. Specifically, the reflectance R of the reflector 220 can be set to any of four levels by changing the proportion of the filler between four levels. The greater the amount of the filler is, the closer the acoustic impedance approaches the acoustic impedance of the filler.

The ultrasonic measurement sheet 200 may include multiple reflector groups 230 that are aligned in the ultrasound transmissive medium 210 as the reflectors 220. Each of the reflector groups 230 includes 1st to p-th (p being an integer greater than or equal to 2) reflectors that are aligned along the depth direction (Z direction) of the ultrasonic measurement sheet 200. The reflector group 230 shown in FIG. 14B includes first to fourth reflectors 220-1 to 220-4, for example. Each reflector group 230 can record code information using the first to fourth reflectors 220-1 to 220-4 included therein. The processing unit 130 performs processing for analyzing the code information recorded using the first to fourth (in a broad sense, p-th) reflectors 220-1 to 220-4.

The same code information may be recorded by each of the reflector groups 230. For example, the same code information may be recorded by all of the reflector groups 230 shown in FIG. 14A. The processing unit 130 performs processing for analyzing the code information recorded by at least one reflector group among the reflector groups 230. According to this configuration, the processing unit 130 can acquire the same code information regardless of which portion of the ultrasonic measurement sheet 200 the ultrasonic probe 300 comes into contact with.

FIG. 15A shows an example of code information recorded using the reflectance of the reflectors 220. FIG. 15A shows an ultrasonic image (B mode image) of a reflector group 230 made up of four reflectors 220. The reflectance of each reflector 220 is set to any of four levels of reflectances R1, R2, R3, and R4 (R1<R2<R3<R4). The reflectance R4 is the highest, and the reflectance R1 is the lowest. In the B mode image that is obtained, the luminance is higher the higher the reflectance of the subject is, and therefore the image of the reflector 220 with the reflectance R1 has the lowest luminance, and the luminances of the images of the reflectors 220 rise in correspondence with the rising reflectances R2, R3, and R4. The processing unit 130 can analyze the code information recorded by the reflector group 230 through obtaining the luminance of each of the reflectors 220 based on ultrasonic image data.

For each image of a reflector 220, the processing unit 130 determines which of the four luminance levels the luminance (luminance information) of the image corresponds to. A luminance level d is then obtained for each reflector 220 based on the determination results. The luminance level d takes any value among the values “0”, “1”, “2”, and “3”. Next, the processing unit 130 obtains the code information a based on the luminance levels d of the reflectors 220.

FIG. 15B shows an example of a luminance table indicating the correspondence between the luminance level d and image luminance. In FIG. 15B, the reflector 220 luminances are shown as relative values based on the maximum value of “100”. For example, if the luminance of a certain reflector 220 is in the range of “21” to “40”, the luminance level d of that reflector 220 is “0”. Also, if the luminance of a certain reflector 220 is in the range of “61” to “80”, the luminance level d of that reflector 220 is “2”. In this way, the processing unit 130 can obtain the luminance level d for each reflector 220.

In the example shown in FIG. 15A, the luminance levels d1 to d4 of the four reflectors 220 included in the reflector group 230 are d1=0, d2=1, d3=2, and d4=3 in order along the bz direction (depth direction). The processing unit 130 obtains the code information a using the following equation.

α=4³×d1+4²×d2+4×d3+d4 (6)

For example, in the case shown in FIG. 15A, the code information α is α=27. In this way, the code information α=27 is recorded in the reflector group 230 shown in FIG. 15A.

FIG. 16 shows an example of ultrasonic images of reflector groups 230 recording code information a having the values “0” to “15”. Each reflector group 230 includes four reflectors 220 similarly to the case shown in FIG. 15A.

Letting d1, d2, d3, and d4 be the luminance levels d of the four reflectors 220 in order along the bz direction (depth direction): d1=d2=d3=d4=0 in the case of the code information α=0; d1=d2=0, d3=1, and d4=3 in the case of the code information α=7; and d1=d2=0 and d3=d4=2 in the case of the code information α=10, for example. By setting each of the four reflectors 220 to any one of four levels of reflectances, it is possible to record 4⁴=256 types of code information, that is to say α=0 to 255.

In this way, according to the ultrasonic measurement sheet 200 that has reflector groups, the code information a can be recorded by setting the reflectances of the reflectors 220 included in a reflector group 230 to predetermined values. The processing unit 130 can then acquire the code information a by performing analysis processing based on the luminance (luminance information) of the ultrasonic image of the reflector group 230.

According to the fourth configuration example of the ultrasonic measuring device 100 of this embodiment, the processing unit 130 can perform processing for analyzing code information recorded in the ultrasonic measurement sheet 200 based on the reception signal, and acquire the reference thickness information DA of the ultrasonic measurement sheet based on the code information a. The processing unit 130 can then acquire depth information regarding the interface BS between the test subject and the ultrasonic measurement sheet 200 in ultrasonic measurement based on the reception signal, and acquire inclination information regarding the ultrasonic probe 300 by performing processing for comparing the depth information with the reference thickness information DA of the ultrasonic measurement sheet 200.

As shown in FIGS. 15A, 15B, and 16, in the case where one reflector group 230 includes four reflectors 220, it is possible to record α=0 to 255 as the code information α. A configuration is possible in which a reference table that associates the code information α with the reference thickness information DA of the ultrasonic measurement sheet is stored in advance in a storage unit of the ultrasonic measuring device 100, and the processing unit 130 analyzes the code information a based on the reception signal and acquires the reference thickness information DA that corresponds to the analyzed code information a based on the reference table.

FIG. 17 is an example of a flowchart of processing for analyzing code information in the fourth configuration example of the ultrasonic measuring device 100 of this embodiment. The processing flow shown in FIG. 17 is executed by the processing unit 130.

Firstly, the processing unit 130 sets the scan direction coordinate value bx to the initial value bx=0 (step S51).

Next, the processing unit 130 obtains, from the image data, a luminance L(bx,bz1) of the pixel that corresponds to the scan direction coordinate value bx and a depth value bz1. The processing unit 130 then determines whether or not the luminance L(bx,bz1) is greater than or equal to a prescribed value (step S52). Here, bz1 is the depth bz that corresponds to the reflectors 220 that are at the most shallow position. If the luminance L(bx,bz1) is greater than or equal to the prescribed value, the procedure moves to step S53. Here, the prescribed value is the minimum luminance value of a reflector 220 image for example, and in the example shown in FIG. 15B, the minimum luminance value (relative value) is specifically “21”.

In step S53, the processing unit 130 obtains luminances L(bx,bz1), L(bx,bz2), L(bx,bz3), and L(bx,bz4) of four reflectors 220 having the same scan direction coordinate value bx and different depths bz. Here, the relationship bz1<bz2<bz3<bz4 is satisfied.

Subsequently, the processing unit 130 obtains the luminance levels d1, d2, d3, and d4 that correspond to the luminances L(bx,bz1), L(bx,bz2), L(bx,bz3), and L(bx,bz4) of the four reflectors 220, and furthermore obtains the code information a from the luminance levels d1, d2, d3, and d4 (step S54).

On the other hand, if the luminance L(bx,bz1) is less than the prescribed value, the pixel does not correspond to a reflector 220 image, and therefore the processing unit 130 increments the scan direction coordinate value bx, that is to say bx=bx+1 (step S55). The processing unit 130 then determines whether or not the incremented value of bx is lower than a scan width W (step S56). The scan width W is a value that corresponds to the number of pixels along the scan direction in the ultrasonic image, for example.

If the incremented value of bx is lower than the scan width W, the procedure returns to step S52, and the processing unit 130 determines whether or not the luminance L(bx,bz1) is greater than or equal to the prescribed value. Here, if the luminance is again less than the prescribed value, the processing unit 130 again increments the value of bx. In this way, the processing unit 130 increments the value of bx until the luminance L(bx,bz1) is greater than or equal to the prescribed value, and the processing target pixel moves along the scan direction.

If it is determined that the value of bx incremented in this way is greater than or equal to the scan width W, this procedure ends without code information analysis processing being performed since the processing unit 130 was not able to find a reflector 220 image.

The processing performed by the processing unit 130 for acquiring inclination information regarding the ultrasonic probe 300 will not be described in detail here since it is the same as in the first and second configuration examples described above.

In this way, according to the fourth configuration example of the ultrasonic measuring device 100 of this embodiment, the processing unit 130 can perform processing for analyzing code information, and acquire appropriate reference thickness information that corresponds to the ultrasonic measurement sheet 200 that is to be used. Inclination information regarding the ultrasonic probe can then be acquired using appropriate reference thickness information. As a result, it is possible to prevent the acquisition of incorrect inclination information due to using incorrect reference thickness information.

1-(6) Fifth Configuration Example

FIG. 18 shows an example of a configuration of the ultrasonic measurement sheet 200 used in a fifth configuration example of the ultrasonic measuring device 100 of this embodiment. FIG. 18 is a top view of the ultrasonic measurement sheet 200. The ultrasonic measurement sheet 200 includes the ultrasound transmissive medium 210 and multiple reflector groups 230. Since the ultrasound transmissive medium 210 has been described with reference to FIG. 9, it will not be described in detail here.

As described with reference to FIGS. 15A, 15B, and 16, the reflector groups 230 can record code information a. The code information a recorded by the reflector groups 230 can be associated with sheet coordinates (x,y) of the ultrasonic measurement sheet 200.

As shown in FIG. 18, for example, it is possible to associate the code information a recorded by the reflector groups 230 with sheet coordinates (x,y). The sheet coordinates are coordinates representing the arrangement positions of the reflector groups 230 in the sheet plane of the ultrasonic measurement sheet 200, and as shown in FIG. 18 for example, the sheet coordinates are expressed using an x coordinate value and a y coordinate value, with the arrangement position of the reflector group 230 recording the code information α=0 being (0,0).

The ultrasonic measurement sheet 200 shown in FIG. 18 includes reflector groups 230 that are arranged in a matrix having six rows and six columns. The code information α=0 to 35 corresponding to the sheet coordinates (x,y) as shown in FIG. 18 is recorded by these reflector groups 230. For example, the sheet coordinates of the reflector group 230 recording the code information α=27 are (3,4).

According to the fifth configuration example of the ultrasonic measuring device 100 of this embodiment, the processing unit 130 can obtain the sheet coordinates (x,y) indicating the arrangement position of the reflector group 230 that was analyzed, based on the code information a acquired through the code information analysis processing. Specifically, if the correspondence relationship between the code information a of the reflector groups 230 and sheet coordinates (x,y) is stored in advance as a reference table, the processing unit 130 can obtain the sheet coordinates (x,y) that correspond to the code information a acquired from the ultrasonic image data. It is then possible to specify the location where the ultrasonic probe 300 was brought into contact, that is to say the scan location with respect to the ultrasonic measurement sheet 200, based on the sheet coordinates (x,y) obtained in this way.

For example, if the scan location is the location indicated by SC1 in FIG. 18, the processing unit 130 acquires code information α=0, 1, 2, 3 by performing code information analysis processing based on the B mode image. The processing unit 130 then acquires the sheet coordinates (0,0), (1,0), (2,0), and (3,0) that correspond to the code information α=0, 1, 2, 3 based on the reference table that was stored in advance. In this way, the processing unit 130 can specify the scan location SC1 shown in FIG. 18.

As another example, if the scan location is the location indicated by SC2 in FIG. 18, the processing unit 130 acquires code information α=20, 15, 10 by performing code information analysis processing based on the B mode image. The processing unit 130 then acquires the sheet coordinates (2,3), (3,2), and (4,1) that correspond to the code information α=20, 15, 10 based on the reference table that was stored in advance. In this way, the processing unit 130 can specify the scan location SC2 shown in FIG. 18.

The flow of code information analysis processing performed by the processing unit 130 will not be described in detail here since it is the same as that shown in FIG. 17. Also, the processing performed by the processing unit 130 for acquiring inclination information regarding the ultrasonic probe 300 will not be described in detail here since it is the same as in the first and second configuration examples described above.

In this way, according to the fifth configuration example of the ultrasonic measuring device 100 of this embodiment, the processing unit 130 can analyze the code information recorded in the ultrasonic measurement sheet 200 and specify the scan location of the ultrasonic probe 300 with respect to the ultrasonic measurement sheet 200. Inclination information regarding the ultrasonic probe 300 at the specified scan location can then be acquired. As a result, even a user who is not familiar with operating the ultrasonic measuring device can hold the ultrasonic probe 300 at the appropriate scan location and so as to not be inclined relative to the surface of the test subject while performing ultrasonic measurement.

2. Ultrasonic Image Device

FIGS. 19A and 19B show examples of specific configurations of the ultrasonic image device 400 of this embodiment. FIG. 19A shows a portable ultrasonic image device 400, and FIG. 19B shows a stationary ultrasonic image device 400.

The portable and stationary ultrasonic image devices 400 both include the ultrasonic measuring device 100, the ultrasonic probe 300, a cable 350, and the display unit 410. The ultrasonic probe 300 includes the ultrasonic transducer device 310 and is connected to the ultrasonic measuring device 100 via the cable 350. The display unit 410 displays display image data.

At least a portion of the emission unit 110, the reception unit 120, and the processing unit 130 of the ultrasonic measuring device 100 can be provided in the ultrasonic probe 300.

FIG. 19C shows an example of the specific configuration of the ultrasonic probe 300 of this embodiment. The ultrasonic probe 300 includes a probe head 315 and a probe body 320, and as shown in FIG. 19C, the probe head 315 is detachable from the probe body 320.

The probe head 315 includes the ultrasonic transducer device 310, a probe base 311, a probe housing 312, and a probe head-side connector 313.

The probe body 320 includes a probe body-side connector 323. The probe body-side connector 323 is connected to the probe head-side connector 313. The probe body 320 is connected to the ultrasonic measuring device 100 via the cable 350. Note that at least a portion of the emission unit 110 and the reception unit 120 of the ultrasonic measuring device 100 can be provided in the probe body 320.

Note that although various embodiments have been explained in detail above, a person skilled in the art will readily appreciate that it is possible to implement numerous variations and modifications that do not depart substantially from the novel aspects and effect of the invention. Accordingly, all such variations and modifications are also to be included within the scope of the invention. For example, terms that are used within the description or drawings at least once together with broader terms or alternative synonymous terms can be replaced by those other terms at other locations as well within the description or drawings. Also the configuration and operation of the ultrasonic measuring device and the ultrasonic image device are not limited to those described in the embodiments, and various modifications are possible.

The entire disclosure of Japanese Patent Application No. 2013-53229, filed Mar. 15, 2013 is expressly incorporated by reference herein.

What is claimed is:

1. An ultrasonic measuring device comprising:
an emission unit that performs ultrasound emission processing;
a reception unit that performs ultrasonic echo reception processing; and
a processing unit that performs ultrasonic measurement control processing,
wherein the processing unit acquires inclination information regarding an ultrasonic probe based on a reception signal resulting from ultrasonic echoes from an interface between a test subject and an ultrasonic measurement sheet, or resulting from ultrasonic echoes from the ultrasonic measurement sheet.

2. The ultrasonic measuring device according to claim 1,
wherein the processing unit acquires depth information regarding the interface in ultrasonic measurement based on the reception signal, and acquires the inclination information based on the depth information.

3. The ultrasonic measuring device according to claim 2,
wherein the processing unit acquires the inclination information by performing processing for comparing the depth information with reference thickness information of the ultrasonic measurement sheet.

4. The ultrasonic measuring device according to claim 3,
wherein the depth information is information regarding depth in an ultrasound emission direction from a face of the ultrasonic measurement sheet that opposes the ultrasonic probe to the interface between the ultrasonic measurement sheet and the test subject.

5. The ultrasonic measuring device according to claim 3,
wherein the processing unit specifies the interface by performing processing for comparing an amplitude value in an A mode waveform that is based on the reception signal with a predetermined threshold value.

6. The ultrasonic measuring device according to claim 3,
wherein the processing unit specifies the interface by performing processing for comparing a luminance value in a B mode image that is based on the reception signal with a predetermined threshold value.

7. The ultrasonic measuring device according to claim 3,
wherein the processing unit calculates a luminance variance for a plurality of depths in a B mode image that is based on the reception signal, and specifies the interface between the ultrasonic measurement sheet and the test subject based on the calculated luminance variances.

8. The ultrasonic measuring device according to claim 3,
wherein the processing unit performs processing for analyzing, based on the reception signal, code information that is recorded in the ultrasonic measurement sheet, and acquires the reference thickness information of the ultrasonic measurement sheet based on the code information.

9. The ultrasonic measuring device according to claim 3,
wherein the processing unit performs processing for acquiring, based on the reception signal, code information that is recorded in the ultrasonic measurement sheet, and specifies a scan location of the ultrasonic probe with respect to the ultrasonic measurement sheet based on the acquired code information, and
the processing unit acquires the inclination information regarding the ultrasonic probe at the specified scan location based on the reception signal.

10. The ultrasonic measuring device according to claim 8,
wherein the ultrasonic measurement sheet has an ultrasound transmissive medium and a plurality of reflectors embedded in the ultrasound transmissive medium,
the code information is recorded using at least one of the reflectance, the number, the shape, and the size of the plurality of reflectors,
the reception unit performs processing for receiving an ultrasonic echo from the plurality of reflectors, and outputs a reception signal to the processing unit, and
the processing unit performs processing for analyzing the code information based on the reception signal from the reception unit.

11. The ultrasonic measuring device according to claim 1,
wherein the ultrasonic measurement sheet has an ultrasound transmissive medium and a plurality of reflectors arranged in an array in the ultrasound transmissive medium,
the reception unit performs processing for receiving an ultrasonic echo from the plurality of reflectors, and outputs a reception signal to the processing unit, and
the processing unit analyzes the number of reflections by the plurality of reflectors based on the reception signal, and acquires the inclination information regarding the ultrasonic probe based on the number of reflections.

12. The ultrasonic measuring device according to claim 1,
wherein in a case of determining based on the acquired inclination information that the ultrasonic probe is inclined, the processing unit generates and outputs notification data for notification of the fact that the ultrasonic probe is inclined.

13. An ultrasonic image device comprising:
the ultrasonic measuring device according to claim 1; and
a display unit that displays display image data.

14. A measuring method comprising the steps of:
emitting ultrasound toward a test subject via an ultrasonic measurement sheet;
receiving an ultrasonic echo from the test subject and the ultrasonic measurement sheet; and
acquiring inclination information regarding an ultrasonic probe based on a reception signal resulting from ultrasonic echoes from an interface between the test subject and the ultrasonic measurement sheet, or resulting from ultrasonic echoes from the ultrasonic measurement sheet.
